(19) United States
(12) Patent Application Publication
(10) Pub. No.: US 2016/0310595 A1
(43) Pub. Date: Oct. 27, 2016

(54) MUCOADHESIVE DENTAL GEL INCLUDING APIS

(71) Applicant: Professional Compounding Centers of America (PCCA), Houston, TX (US)

(72) Inventor: TSU-I CATHERINE WANG, SUGAR LAND, TX (US)

(21) Appl. No.: 15/136,563

(22) Filed: Apr. 22, 2016

Related U.S. Application Data
(60) Provisional application No. 62/151,242, filed on Apr. 22, 2015.

(51) Int. Cl. A61K 47/10; A61K 9/06; A61K 9/00

(52) U.S. Cl. CPC A61K 47/10; A61K 9/006; A61K 9/06

(57) ABSTRACT

Formulations for mucoadhesive dental gel compositions including antibiotics are disclosed.

### CROSS-REFERENCE TO RELATED CASES

This application claims the benefit of U.S. provisional patent application Ser. No. 62/151,242, filed on Apr. 22, 2015, and incorporates such provisional application by reference into this disclosure as if fully set out at this point.

### FIELD OF THE INVENTION

The present disclosure relates generally to pharmaceutical compositions, and more particularly, to mucoadhesive dental gels.

### BACKGROUND OF THE INVENTION

Antibiotics are a group of medicines that are used to treat infections caused by germs (e.g., bacteria and certain parasites) and in some cases prevent bacterial infections. Antibiotics are sometimes called antibacterials or antimicrobials and can be used to treat relatively mild conditions, such as acne, as well as potentially life-threatening conditions, such as pneumonia. Antibiotics can be taken by mouth as liquids, tablets, capsules, or they can be given by injection. Antibiotics are also available as creams, ointments, or lotions to apply to the skin to treat certain skin infections. Additionally, antibiotics are often classified into six groups: penicillins, cephalosporins, aminoglycosides, tetracyclines, macrolides, and fluoroquinolones.

Antibacterial action of antibiotics generally falls within one of four mechanisms, three of which involve the inhibition or regulation of enzymes occurred in the cell wall biosynthesis, nucleic acid metabolism and repair, or protein synthesis, respectively. The fourth mechanism involves the disruption of membrane structure. Many of these cellular functions targeted by antibiotics are most active in multiplying cells. Since there is often overlap in these functions between prokaryotic bacterial cells and eukaryotic mammalian cells, some antibiotics have also been found to be useful as anticancer agents.

The stability of an antibiotic depends on its chemical structure, method of isolation (from natural sources or chemical synthesis), and the mechanisms of inactivation. Most antibiotics are stable as dry powders. Dissolving the powder in solvents such as water exposes the compound to hydrolysis, thereby making the antibiotic unstable. Commonly, the instability is reduced by storing the antibiotics in aliquots at −20° C. In an example, penicillin-derived antibiotics and tetracyclines are stable only for 3 months, even stored at −20° C.

Currently, a variety of oral antibiotics are used for treating a plurality of infections. Some examples of these antibiotics are doxycycline, minocycline, tetracycline, oxytetracycline, and the like. However, the systemic administration of antibiotics has only variable success in treating many infections.

Treating periodontal infection by administering antibiotics to the oral cavity is generally ineffective because the periodontal pocket is essentially inaccessible. Dental gel preparations including antibiotics (e.g., tetracyclines), are currently available for the treatment of some infectious diseases, but many of them require premixing of the APIs with an aqueous gel prior to administration due to the instability of antibiotics in aqueous gel formulations. Most of these gel preparations, once in contact with the oral mucosa, tend to melt quickly at body temperature, liquefying soon after application and being washed away from the site of administration before performing the expected pharmacological action. Additionally, gel preparations including antibiotics are only effective for a short period of time after manufacturing due to the short shelf life which is in turn caused by instability of the antibiotics within the preparation.

### SUMMARY OF THE INVENTION

The present disclosure is directed towards mucoadhesive dental gel compositions that include antibiotics as APIs within an anhydrous base, which comprises hydrophilic polymers dispersed in an anhydrous medium, and in combination with optional transmucosal absorption enhancers. These mucoadhesive dental gel compositions provide improved chemical stability for the antibiotics, thereby exhibiting a longer shelf life as compared to conventional aqueous dental gels including said antibiotics. The aforementioned mucoadhesive dental gel compositions are proposed to treat bacterial infections of the gingiva in mammals.

In some embodiments, the hydrophilic polymers dispersed in the anhydrous medium are paste-like dosage forms. When placed in the oral cavity, the hydrophilic polymers will absorb the moisture in the oral cavity, become hydrated and form a gel in situ. In these embodiments, the gel will adhere to the oral mucosa at the site of administration (e.g., periodontal pocket or gingiva) and release the APIs.

In some embodiments, the mucoadhesive dental gel compositions are particularly useful for treating periodontal diseases. In these embodiments, the mucoadhesive dental gel compositions are used to deliver an antibiotic agent to a patient's gingiva, specifically to the periodontal pocket for treating or preventing gingivitis or other periodontal diseases. Further to these embodiments, the mucoadhesive dental gel compositions allow lower dosages of APIs and provide higher concentrations of the APIs at the infection site.

In some embodiments, mucoadhesive dental gel compositions include one or more antibiotics as APIs, one or more hydrophilic polymers dispersed in an anhydrous medium, optional transmucosal absorption enhancers, optional surfactants, other vehicles, and additives, among other suitable ingredients.

In some embodiments, APIs include antibiotics, such as, penicillins, cephalosporins, aminoglycosides, tetracyclines, macrolides, fluoroquinolones, and the like. In other embodiments, the antibiotic employed in mucoadhesive dental gel compositions is doxycycline, minocycline, tetracycline, oxytetracycline, analogs, derivatives, mixtures thereof, or the like.

In some embodiments, the hydrophilic polymers used within the mucoadhesive dental gel compositions are: a polyoxyethylene, such as PEG-90M (e.g., Polyox WSR-301™) and a hydroxypropyl methylcellulose (HPMC), such as Methocel K100M™. In other embodiments, other suitable hydrophilic polymers can be used within the mucoadhesive dental gel compositions. In these embodiments, the suitable hydrophilic polymers include other polyoxyethylenes, hydroxyethyl cellulose, other Methocel™, and the like.

In some embodiments, the mucoadhesive dental gel compositions include an anhydrous medium. In these embodiments, the anhydrous medium provides an environment in which the antibiotic is chemically stable. Further to these embodiments, the anhydrous medium allows longer shelf life of the dental gel.

In some embodiments, the anhydrous medium comprises a mixture of polyethylene glycol compounds (PEGs) with different molecular weight. In these embodiments, the ratio between the compounds within the anhydrous medium can be experimentally determined to obtain a cream consistency. Further to these embodiments, the anhydrous medium within the mucoadhesive dental gel compositions includes a liquid PEG (e.g., PEG 200, PEG 300, PEG 400, and the like) mixed with a solid PEG (with a molecular weight over 1000).

In other embodiments, the anhydrous medium within mucoadhesive dental gel compositions comprises a suitable oil (e.g., a vegetable oil, mineral oil, medium-chain triglyceride, a combination thereof, and the like) and a waxy substance (e.g., shea butter, cocoa butter, paraffin wax, bees wax, spermaceti, hydrogenated vegetable oils, a combination thereof, and the like). In these embodiments, the waxy substances within the anhydrous medium are used to thicken the oils into a desired cream consistency.

In some embodiments, the mucoadhesive dental gel compositions allow the delivery of antibiotics directly at the site of application, such as the gingiva and periodontal pocket of a patient for treating gingivitis or periodontal infection, bypassing the gastrointestinal tract and the hepatic metabolism and will result in a higher percentage of bioavailability of APIs at the infection site.

Numerous other aspects, features, and benefits of the present disclosure may be made apparent from the following detailed description.

### DETAILED DESCRIPTION OF THE PREFERRED EMBODIMENTS

The present disclosure is described here in detail. Other embodiments may be used and/or other changes may be made without departing from the spirit or scope of the present disclosure. The described embodiments are not meant to limit the subject matter presented here.

### Definitions

As used here, the following terms have the following definitions:

“Active Pharmaceutical Ingredients (APIs)” refer to chemical compounds that induce a desired effect, and include agents that are therapeutically or prophylactically effective.

“Absorption Enhancer” or, equivalently, “Penetration Enhancer” refers to a substance used to increase the rate of permeation through the mucous membrane, skin or other body tissue of one or more substances (e.g., APIs) in a formulation.

“Periodontal Pocket” refers to a pathologic deepening of the gingival sulcus resulting from detachment of the gingiva from the tooth.

“Treating” and “Treatment” refers to reduction in severity and/or frequency of symptoms, elimination of symptoms and/or underlying cause, prevention of the occurrence of symptoms and/or their underlying cause, and improvement or remediation of damage.

“Vehicle” refers to a substance of no therapeutic value that is used to convey at least one API for administration.

### Description of the Disclosure

The present disclosure is directed towards mucoadhesive dental gel compositions that include antibiotics as APIs within an anhydrous base, which comprises hydrophilic polymers dispersed in an anhydrous medium, and in combination with optional transmucosal absorption enhancers. These mucoadhesive dental gel compositions provide improved chemical stability for the antibiotics, thereby exhibiting a longer shelf life as compared to conventional aqueous dental gels including said antibiotics. The aforementioned mucoadhesive dental gel compositions are proposed to treat bacterial infections of the gingiva in mammals.

In some embodiments, the hydrophilic polymers dispersed in the anhydrous medium are paste-like dosage forms. When placed in the oral cavity, the hydrophilic polymers will absorb the moisture in the oral cavity, become hydrated and form a gel in situ. In these embodiments, the gel will adhere to the oral mucosa at the site of administration (e.g., periodontal pocket or gingiva) and release the APIs.

In some embodiments, the mucoadhesive dental gel compositions are particularly useful for treating periodontal diseases. In these embodiments, the mucoadhesive dental gel compositions are used to deliver an antibiotic agent to a patient's gingiva, specifically to the periodontal pocket for treating or preventing gingivitis or periodontal diseases. Further to these embodiments, the mucoadhesive dental gel compositions allow lower dosages of APIs and provide higher concentrations of the APIs at the infection site.

In other embodiments, the mucoadhesive dental gel compositions are used for treating a plurality of bacterial infections in the gingiva. In these embodiments, the APIs to be used within the mucoadhesive dental gel compositions depend on the bacteria causing the infection on the patient.

### Formulation

Mucoadhesive dental gel compositions include one or more antibiotics as APIs, one or more hydrophilic polymers dispersed in an anhydrous medium, optional transmucosal absorption enhancers, optional surfactants, other vehicles and additives among other suitable ingredients.

According to some embodiments, APIs include antibiotics, such as penicillins, cephalosporins, aminoglycosides, tetracyclines, macrolides, fluoroquinolones, and the like.

In some embodiments, the antibiotic employed within mucoadhesive dental gel compositions is doxycycline, minocycline, tetracycline, oxytetracycline, the analogs, derivatives, or mixtures thereof, or the like. The list of antibiotics above is not exhaustive; other compounds described in the art that meet the set requirements can also be considered.

In some embodiments, the hydrophilic polymers used within the mucoadhesive dental gel compositions are: a polyoxyethylene, such as PEG-90M (e.g., Polyox WSR-301™) and a hydroxypropyl methylcellulose (HPMC), such as Methocel K100M™. In other embodiments, other suitable hydrophilic polymers can be used within the mucoadhesive dental gel compositions. In these embodiments, the suitable hydrophilic polymers include other polyoxyethylenes, hydroxyethyl cellulose, other Methocel™, and the like.

In some embodiments, the mucoadhesive dental gel compositions include an anhydrous medium. In these embodiments, the anhydrous medium provides an environment in which the antibiotic is chemically stable. Further to these embodiments, the anhydrous medium allows longer shelf life of the dental gel.

In some embodiments, the anhydrous medium comprises a mixture of polyethylene glycol compounds (PEGs) with different molecular weight. In these embodiments, the ratio between the compounds within the anhydrous medium can be experimentally determined to obtain a creamy consistency. Further to these embodiments, the anhydrous medium within the mucoadhesive dental gel compositions includes a liquid PEG (e.g., PEG 200, PEG 300, PEG 400, and the like) mixed with a solid PEG (with a molecular weight over 1000).

In other embodiments, the anhydrous medium within mucoadhesive dental gel compositions comprises a suitable oil (e.g., a vegetable oil, mineral oil, medium-chain triglyceride, a combination thereof, and the like) and a waxy substance (e.g., shea butter, cocoa butter, paraffin wax, bees wax, spermaceti, hydrogenated vegetable oils, a combination thereof, and the like). In these embodiments, the waxy substances within the anhydrous medium are used to thicken the oils into a desired creamy consistency.

In some embodiments, various additives are included to facilitate the preparation of suitable dosage forms. For example, additives include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants, among others.

In some embodiments, the mucoadhesive dental gel compositions include a sweetening agent, such as sucrose or saccharin, among others; natural or artificial flavors, such as peppermint, methyl salicylate, or orange flavor, among others.

The pH adjusting agents include sodium bicarbonate, magnesium hydroxide, calcium carbonate, dibasic calcium phosphate, tribasic calcium phosphate, sodium bicarbonate, magnesium hydroxide, potassium hydroxide, citric acid, lactic acid, hydrochloric acid, sulfuric acid, phosphoric acid, sodium phosphate monobasic, and sodium phosphate dibasic, among others.

Surfactants include: polysorbates such as polysorbate 20, 40, 60, and 80, among others; sorbitan esters such as sorbitan monolaurate, and sorbitan monopalmitate, sorbitan monooleate, among others; and sodium lauryl sulfate, among others.

In some embodiments, a stabilizing agent is used to stabilize the API for a specific dosage form. In these embodiments, the stabilizing agent used will depend on the API used as well as the other additive ingredients. Any suitable chemical substance may be used as a stabilizing agent. Stabilizing agents are known to those skilled in the art and therefore will not be discussed further herein.

Mucoadhesive polymers include: gums such as acacia, agarose, alginic acid, sodium alginate and other alginic acid derivatives, carrageenan, gelatin, gellan, guar gum, hakea gum, karaya gum, and locust bean gum, among others; chitosan and chitosan derivatives; hyaluronic acid, pectin, and other polysaccharides; gelatin, polyisoprene, polyisobutylene, polyetherurethane, polyvinylalcohol, polyvinylpyrrolidone, polycarbophil, polyethylene oxide polymers, and pullulan, among others. Mucoadhesive polymers also include: cellulose derivatives such as ethyl cellulose, cellulose acetate, hydroxyethyl cellulose, hydroxypropylcellulose, hydroxypropylmethylcellulose, methylcellulose, methylhydroxyethylcellulose, and sodium carboxymethyl cellulose, among others; poly(acrylic acid)-based polymers such as polyacrylates, poly (methylvinylether-co-methacrylic acid), poly(acrylic acid-co-ethylhexylacrylate), poly(acrylic acid-co-acrylamide), poly(acrylic acid-co-butylacrylate), poly(acrylic acid-co-methyl methacrylate), poly (2-hydroxyethyl methacrylate), polymethacrylates, poly(alkylcyanoacrylate) and other cyanoacrylates, poly (isohexycyanoacrylate), poly (isobutylcyanoacrylate), and hydroxyethyl methacrylate, and any other polymer known to a person skilled in the art that exhibits mucoadhesive characters.

In some embodiments, transmucosal absorption enhancers provide more efficient penetration of API through oral mucosal tissue. In these embodiments, the transmucosal absorption enhancers allow lower API dosage requirements.

Oral transmucosal absorption enhancers include: enzyme inhibitors such as aprotinin and puromycin, among others; chitosan and chitosan derivatives such as chitosan glutamate, trimethyl chitosan, chitosan-4-thioglycolic acid, 5-methyl-pyrrolidine chitosan, and chitosan-4-thio-butylamidine, among others; alpha, beta, and gama cyclodextrins such as dimethyl cyclodextrin, sulfobutyl cyclodextrin, 2-hydroxypropyl-beta-cyclodextrin, poly-beta-cyclodextin, and methylated beta-cyclodextrin, among others; bile salts such as sodium deoxycholate, sodium glycocholate, sodium glycodeoxycholate, sodium glycodihydrofusidate, sodium taurocholate, sodium taurodeoxycholate, sodium tauroglycocholate, sodium taurodihydrofusidate, and sodium ursocholate, among others; chelating agents such as sodium EDTA, citric acid, sodium citrate, sodium salicylate, methylsalicylate, methoxysalicylate, and polyacrylates, among others; alcohols such as ethanol and isopropanol, among others; fatty acids and derivatives such as oleic acid, methyloleate, capric acid, neodecanoic acid, elaidic acid, lauric acid, palmitoylearnitine, cod liver oil extract, mono glycerides and diglycerides of oleic acid andcapric acid, lauric acid, sodium laurate, linoleic acid, sodium fusidate, sodium caprate, lyceryl monolaurate, glyceryl monooleate, glyceryl monostearate, sucrose fatty acid esters, and diethylene glycol monoethyl ether, among others; lecithins and phospholipids such as phosphatidylcholine, lysophosphatidyl choline, and didecanoylphophatidylcholine, among others; sulfoxides such as dimethylsulfoxide and decylmethyl sulfoxide, among others; polyols such as glycerin, propylene glycol, propanediol, and polyethylene glycols of various molecular weights, among others; urea and derivatives such as unsaturated cyclic urea, among others; surfactants such as sodium dodecyl sulfate, sodium lauryl sulfate, dioctyl sodium sulfosuccinate, nonylphenoxypolyoxyethylene, polyoxyethylene alkyl ethers, polyoxyethylene-9-lauryl ether, polyoxyethylene 23 lauryl ether, polyoxyethylene-20-cetyl ether, polyethylene glycol dodecyl ether, polyethylene glycol-8 laurate, glyceryl monolaurate, polyoxyethylene stearates, polysorbates, sorbitan fatty acid esters, polyoxyethylene castor oil derivatives, benzalkonium chloride, cetylpyridinium chloride, and cetyltrimethylammonium bromide, among others. Other oral transmucosal absorption enhancers include alkylglycosides, azone, hyaluronic acid, sodium Hyaluronate, glycine chenodeoxycholate, lauroyl macroglycerides, isopropyl myristate, isopropyl palmitate, glutathione, witepsol, menthol, capsaicin, taurine, tocopheryl acetate, lauroyl macroglycerides, lionoleoyl polyoxyl-6 glycerides; diethylene glycol monoethyl ether, dextran sulfate, various saponins, poly-1-arginine, and 1-lysine, and any other chemical known to a person skilled in the art that exhibits penetration enhancing effect on transmucosal absorption.

In some embodiments, amount of absorption enhancers included within mucoadhesive gel compositions range from about 0.1% to about 20%; most suitable amount is of about 1% to about 10%. These percent ranges may refer to % weight by weight, % weight by volume, or % volume by volume.

In some embodiments, the absorption enhancer is chosen to avoiding irritation of the gingiva or other oral tissues. Reduced irritation or non-irritating absorption enhancers may, include, but are not limited to chitosan; alpha, beta, and gama cyclodextrins; fatty acids and derivatives such as oleic acid, methyloleate, capric acid, neodecanoic acid, elaidic acid, lauric acid, palmitoylearnitine, cod liver oil extract, mono glycerides and diglycerides of oleic acid and capric acid, lauric acid, sodium laurate, linoleic acid, sodium fusidate, sodium caprate, lyceryl monolaurate, glyceryl monooleate, glyceryl monostearate, sucrose fatty acid esters; lecithins and phospholipids such as phosphatidylcholine, lysophosphatidyl choline, and didecanoylphophatidylcholine; polyols such as glycerin, polyethylene glycols of various molecular weights; polysorbates, sorbitan fatty acid esters, hyaluronic acid, sodium hyaluronate; diethylene glycol monoethyl ether, poly-1-arginine, and 1-lysine.

In other embodiments, mucoadhesive dental gel compositions include pharmaceutical solvents to produce gel-forming creams or pastes.

In some embodiments, pharmaceutical solvents for liquid dosage forms of mucoadhesive dental gel compositions include liquid polyethylene glycols of various molecular weights, ethyl oleate, medium chain triglycerides, isopropyl myristate, isopropyl palmitate, isopropyl stearate, and other pharmaceutically acceptable esters of C8-C22 fatty acids, mineral oils, and vegetable oils, among others.

C8-C22 fatty acids include fatty acids having from 8 to 22 carbon atoms, such as myristic acid, palmitic acid, stearic acid, arachidic acid, or oleic acid, among others.

Examples of vegetable oils include almond oil, peanut oil, sesame oil, sunflower oil, safflower oil, canola oil, corn oil, and olive oil, among others.

In some embodiments, the mucoadhesive dental gel compositions include petrolatum, PCCA Plasticized™ base, paraffin wax, various synthetic wax, lanolin, beeswax, carnauba wax, candelila wax, silicones, isopropylesters, polyols, cellulose ethers, among other suitable bases. In addition, ointment bases also include suitable pharmaceutical solvents, such as liquid polyethylene glycols of various molecular weights, ethyl oleate, medium chain triglycerides, isopropyl myristate, isopropyl palmitate, isopropyl stearate, and other pharmaceutically acceptable esters of C8-C22 fatty acids and C2-C6 alcohols, mineral oils, and vegetable oils, among others.

### Administration

In some embodiments, the mucoadhesive dental gel compositions allow the delivery of antibiotics directly at the infection sites, such as the gingiva and periodontal pocket of a patient, bypassing the gastrointestinal tract and the hepatic metabolism and will result in a higher percentage of bioavailability of APIs at the infection site.

In some embodiments, the mucoadhesive dental gel compositions are administered in the oral cavity at the sublingual, palatal, buccal, gingival, or the like. In these embodiments, mucoadhesive dental gel compositions may be self-administered by the patient or administered by a medical practitioner, such as a physician or nurse.

In some embodiments, the concentrations of the APIs in the dosage forms, required dosages, dosing frequency, and period of treatment will depend on the severity of the infection, size of infected area, and the type of antibiotic included in the disclosed mucoadhesive dental gel compositions. In other words, some antibiotics are more potent than others and some antibiotics are used to treat certain types of infections; hence, the dosage regimen can be adjusted as needed for the infection as recommended by the physician.

While various aspects and embodiments have been disclosed, other aspects and embodiments are contemplated. The various aspects and embodiments disclosed are for purposes of illustration and are not intended to be limiting, with the true scope and spirit being indicated by the following claims.

It should also be understood that in some embodiments, the ingredients and/or steps explicitly disclosed constitute the entire composition, formulation, or treatment. In other words, no other chemicals or substances are present in the formulation or composition in any detectable amount. Moreover, the treatment would be explicitly as described without intervening steps or administrations, and the steps would be performed in the prescribed order. Other embodiments comprise the explicitly disclosed ingredients or steps but may also include other ingredients, steps, or order of steps that do not unduly compromise or alter the efficacy or operation of the composition, formulation, or treatment. In further embodiments, the compositions, formulations, and steps disclosed herein may form the basis of additional developments, formulations, or treatments, even if they alter the operation of the disclosed formulation, composition, or treatment method.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26 -100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Thus, the present invention is well adapted to carry out the objects and attain the ends and advantages mentioned above as well as those inherent therein. While the inventive device has been described and illustrated herein by reference to certain preferred embodiments, various changes and further modifications, apart from those shown or suggested herein, may be made therein by those of ordinary skill in the art, without departing from the spirit of the inventive concept the scope of which is to be determined by the following claims.

What is claimed is:

1. A composition comprising:
an anhydrous base including a hydrophilic polymer dispersed in an anhydrous medium; and
an antimicrobial active pharmaceutical within the anhydrous base.

2. The composition of claim 1, further comprising a transmucosal absorption enhancer.

3. The composition of claim 2, wherein the transmucosal absorption enhancer is non-irritant.

4. The composition of claim 2, wherein the transmucosal absorption enhancer comprises an enzyme inhibitor.

5. The composition of claim 2 wherein the transmucosal absorption enhancer comprises an alcohol.

6. The composition of claim 1, wherein the transmucosal absorption enhancer comprises about 0.1% to about 20% of the composition by weight.

7. The composition of claim 6, wherein the transmucosal absorption enhancer comprises about 1% to about 10% of the composition by weight.

8. The composition of claim 1, wherein the hydrophilic polymer is a polyxyethylene.

9. The composition of claim 1, further comprising a pharmaceutical solvent rendering the composition into a gel or paste.

10. The composition of claim 1, further comprising a thickener.

11. A composition comprising:
an anhydrous base prepared as a paste and including a hydrophilic polymer dispersed in an anhydrous medium;
an antimicrobial active pharmaceutical within the anhydrous base; and
a transmucosal absorption enhancer that is non-irritant to the gingiva.

12. The composition of claim 11, wherein the transmucosal absorption enhancer comprises an enzyme inhibitor.

13. The composition of claim 11, wherein the transmucosal absorption enhancer comprises about 0.1% to about 20% of the composition by weight.

14. The composition of claim 13, wherein the transmucosal absorption enhancer comprises about 1% to about 10% of the composition by weight.

15. The composition of claim 13, wherein the hydrophilic polymer is a polyxyethylene.

16. The composition of claim 14, further comprising a pharmaceutical solvent rendering the composition into a gel or paste.

17. A method comprising:
preparing a paste as an anhydrous base that includes a hydrophilic polymer dispersed in an anhydrous medium;
incorporating an antimicrobial active pharmaceutical within the anhydrous base;
incorporating a transmucosal absorption enhancer within the anhydrous base; and
applying the anhydrous base containing the antimicrobial active pharmaceutical and the transmucosal absorption enhancer within the oral cavity of a patient for treatment of infection.

18. The method of claim 17, further comprising selecting the transmucosal absorption enhancer to minimize irritation of the oral cavity.

19. The method of claim 18, further comprising incorporating the transmucosal absorption enhancer to comprises about 0.1% to about 20% of the total anhydrous base containing the antimicrobial active pharmaceutical and the transmucosal absorption enhancer within the oral cavity of a patient for treatment of infection

20. The method of claim 19, further comprising incorporating the transmucosal absorption enhancer to comprises about 1% to about 10% of the total anhydrous base containing the antimicrobial active pharmaceutical and the transmucosal absorption enhancer within the oral cavity of a patient for treatment of infection.
